(19) United States
(12) Patent Application Publication
(10) Pub. No.: US 2004/0142933 A1
(43) Pub. Date: Jul. 22, 2004

(54) Oxindole substituted piperazine derivatives

(76) Inventors: George William Forrest, Vancouver (CA); Harriet Wall Hamilton, Chelsea, MI (US)

Correspondence Address: WARNER-LAMBERT COMPANY, 2800 PLYMOUTH RD, ANN ARBOR, MI, 48105, US

(21) Appl. No.: 10/695,594

(22) Filed: Oct. 28, 2003

Related U.S. Application Data
(60) Provisional application No. 60/421,707, filed on Oct. 28, 2002.

(51) Int. Cl. A61K 31/502; A61K 31/496; C07D 417/14; C07D 413/14; C 07D 4 3/14

(52) U.S. Cl. 514/248; 544/363; 544/237; 544/360; 544/388; 514/253.05; 514/253.09; 514/254.02; 514/254.06

(57) ABSTRACT

This invention relates to compounds of the formula I

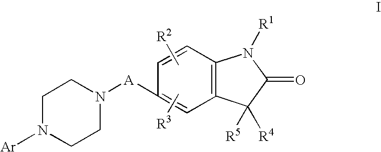

wherein Ar, A, R, R¹, R², R³, R⁴ and R⁵ are defined as in the specification, pharmaceutical compositions containing them and their use in the treatment of central nervous system disorders.

### BACKGROUND OF THE INVENTION

This invention relates to oxindole substituted piperazine derivatives, pharmaceutical compositions containing them and their use for the treatment of schizophrenia and other central nervous system (CNS) disorders.

The oxindole substituted piperazine derivatives of this invention exhibit activity as antagonists of dopamine D2 receptors and of serotonin 2A (5HT2A) receptors.

Other heterocyclic piperazine derivatives that are useful for the treatment of schizophrenia are referred to in U.S. Pat. No. 5,350,747, which issued on Sep. 27, 1994, and in U.S. Pat. No. 6,127,357, which issued on Oct. 3, 2000. These patents are incorporated herein by reference in their entireties.

Other piperazine and piperidine derivatives that have been stated to be useful as antipsychotic agents are those referred to in PCT patent publication WO 93/04684, which published on Mar. 18, 1993, and European patent application EP 402644A, which was published on Dec. 19, 1990. These patent applications are incorporated herein by reference in their entireties.

### SUMMARY OF THE INVENTION

The present invention relates to compounds of the formula I

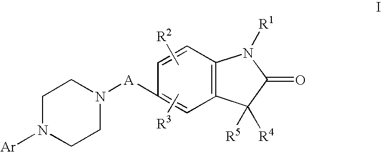

wherein Ar is 1,2-benzisothiazoyl, 1,2-benzisothiazoyl-1-oxide, 1,2-benzisothiazoyl-1-dioxide, 1,2-benzisoxazoyl, naphthyl, pyridyl, quinolyl, isoquinolyl, benzothiadiazolyl, benzotriazolyl, benzoxazolyl, benzoxazolonyl, phthalazinyl, indolyl, indanyl, 1H-indazoyl, or 3-indazolyl, and wherein Ar can optionally be substituted by one or more substituents, preferably from zero to three substituents, independently selected from halo, preferably chloro or fluoro, cyano, nitro, (C₁-C₆) alkyl optionally substituted with from one to three fluorine atoms and (C₁-C₆) alkoxy optionally substituted with from one to three fluorine atoms; with the proviso that Ar can not be attached to the piperazine ring via a phenyl ring of Ar;

A is —(CH₂)ₙCH₂—, wherein n is an integer from one to three, wherein one of the CH₂ groups of A that is not adjacent to the piperazine nitrogen can optionally be replaced by an oxygen atom or by NR, wherein R is (C₁-C₆) alkyl, and wherein one of the carbon atoms of A can optionally be substituted by oxo, amino, NHR wherein R is hydroxy or (C₁-C₆) alkyl, and wherein each R group in a compound of the formula I is independent of any other R group in such compound;

R² and R³ are independently selected from hydrogen, (C₁-C₆) alkyl optionally substituted with from one to three fluorine atoms, (C₁-C₆) alkoxy optionally substituted with from one to three fluorine atoms, (C₂-C₆) alkenyl optionally substituted with from one to three fluorine atoms, (C₂-C₆) alkenoxy optionally substituted with from one to three fluorine atoms, —C(C═O)—(C₁-C₆)alkyl, —C(C═O)—(C₁-C₆)alkenyl which can have one or two sites of unsaturation, halogen, nitro, cyano, hydroxy, amino, (C₁-C₆) alkylamino, di-(C₁-C₆) alkylamino, aryl and heteroaryl, and wherein said aryl and heteroaryl groups can optionally be substituted with one or more substituents, preferably from zero to two substituents, independently selected from halo, oxo, nitro, amino, cyano, (C₁-C₆) alkyl optionally substituted with from one to three fluorine atoms and (C₁-C₆) alkoxy optionally substituted with from one to three fluorine atoms;

R¹ is hydrogen, (C₁-C₄) alkyl optionally substituted with from one to three fluorine atoms, aryl, —C(O)R⁶ wherein R⁶ is aryl, (C₁-C₄) alkyl, or aryl-(C₁-C₄) alkyl-, and wherein the alkyl moieties of the aryl-(C₁-C₄) alkyl- and heteroaryl-(C₁-C₄) alkyl groups can be optionally substituted with from one to three fluoro atoms, and wherein the aryl and heteroaryl moieties of these groups can optionally be substituted with one or more substituents, preferably with from zero to two substituents, independently selected from halo, nitro, amino, cyano, (C₁-C₆) alkyl optionally substituted with from one to three fluorine atoms and (C₁-C₆) alkoxy optionally substituted with from one to three fluorine atoms;

R⁴ and R⁵ together represent an olefin optionally terminally substituted by one or two substituents, R⁷ and R⁸, which are independently selected from the group of substituents set forth above in the definition of R² and R³;

or R⁴ and R⁵, taken together, can form a spiro saturated ring containing from 3 to 6 carbon atoms, wherein said ring can be optionally substituted by one or two substituents, R⁷ and R⁸, which are independently selected from the group of substituents set forth above in the definition of R² and R³;

with the proviso that when Ar is benzisothiazol-3-yl, and A is ethylene, and R¹ is hydrogen or unsubstituted (C₁-C₄)alkyl, and R² is hydrogen, halo or methyl, and R³ is hydrogen, halo, nitro, amino, cyano, or substituted or unsubstituted alkyl or substituted or unsubstituted alkoxy; then R⁴ and R⁵ cannot form either a spiro (C₄-C₆)cycloalkyl group or an olefin terminally substituted with R⁷ and R⁸ wherein R⁷ is hydrogen and R⁸ is phenyl;

and the pharmaceutically acceptable salts of such compounds.

Preferred compounds of this invention include compounds of the formula I wherein Ar is a bicyclic ring system selected from the following:

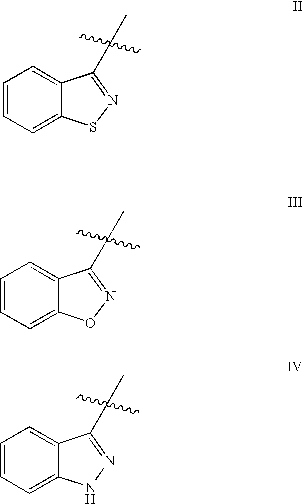

wherein ring systems II, III and IV can be optionally substituted as described above in the definition of formula I and wherein A is —CH₂—, —CH₂—CH₂—, —(C═O)—, —CH₂(C═O)—, —CH(OH)—, —CH₂—CH(OH)—, —CH—N(R)—, or —CH₂—CH—N(R)—, and wherein the oxindole moiety attached to A is selected from the following:

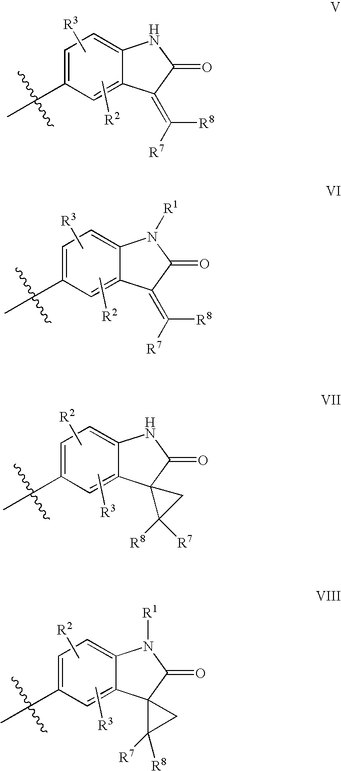

wherein R¹, R² and R³ are as defined above and wherein the spirocyclopropyl groups can be substituted or unsubstituted.

Preferred compounds of the invention include the following compounds and their pharmaceutically acceptable salts:

5-[2-(4-1,2-Benzisothiazol-3-yl-piperazin-1-yl)-ethyl]-3-isopropylidene-1-methyl-1,3-dihydro-indol-2-one;

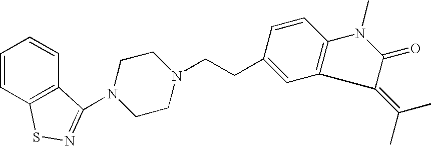

5-[2-(4-1,2-Benzisothiazol-3-yl-piperazin-1-yl)-ethyl]-3-isopropylidene-1,3-dihydro-indol-2-one;

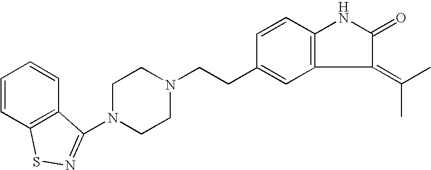

5-[3-(4-1,2-Benzisothiazol-3-yl-piperazin-1-yl)-propyl]-3-isopropylidene-1,3-dihydro-indol-2-one;

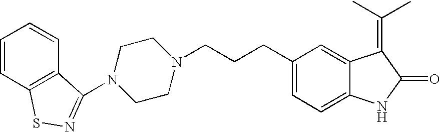

5-[3-(4-1,2-Benzisoxazol-3-yl-piperazin-1-yl)-propyl]-3-isopropylidene-1,3-dihydro-indol-2-one;

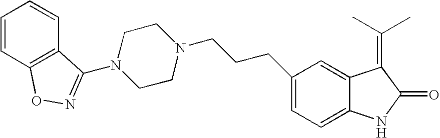

5-[2-(4-1,2-Benzisothiazol-3-yl-piperazin-1-yl)-ethyl]-6-chloro-3-isopropylidene-1,3-dihydro-indol-2-one;

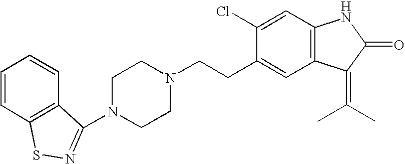

5-[2-(4-1,2-Benzisothiazol-3-yl-piperazin-1-yl)-propyl]-6-chloro-3-isopropylidene-1,3-dihydro-indol-2-one;

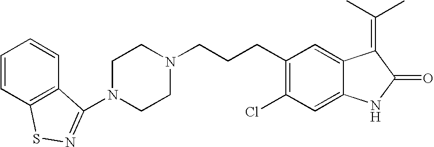

5-[3-(4-1,2-Benzisoxazol-3-yl-piperazin-1-yl)-ethyl]-3-isopropylidene-1,3-dihydro-indol-2-one;

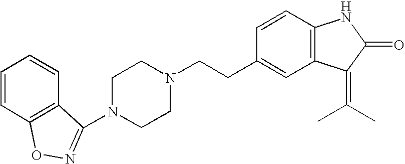

5-{3-[4-(1H-Indazol-3-yl)-piperazin-1-yl]-propyl}-3-isopropylidene-1,3-dihydro-indol-2-one;

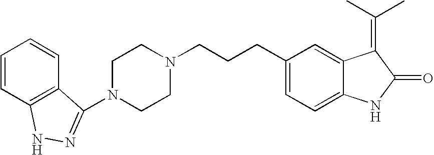

Spiro[cyclopropane-1,3′-{3H}indol]-2′(1′H)-one,5′-[2-[4-{1,2-benzisothiazol-3-yl}-1-piperazinyl]ethyl]-1′,2,2-trimethyl—

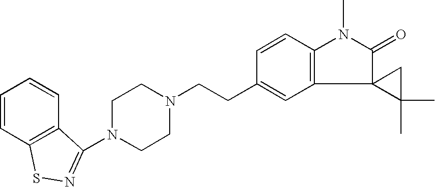

Spiro[cyclopropane-1,3′-{3H}indol]-2′(1′-one,5′-[2-[4-{1,2-benzisothiazol-3-yl}-1-piperazinyl]ethyl]-2,2-dimethyl—

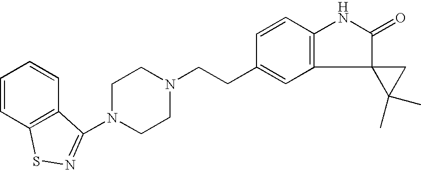

Spiro[cyclopropane-1,3′-{3H}indol]-2′(1═H)-one,5′-[3-[4-{1,2-benzisothiazol-3-yl}-1-piperazinyl]propyl]-2,2-dimethyl—

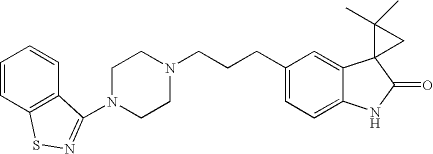

Spiro[cyclopropane-1,3′-{3H}indol]-2′(1′H)-one,5′-[2-[4-{1,2-benzisothiazol-3-yl}-1-piperazinyl]ethyl]-6′-chloro-2,2-dimethyl—

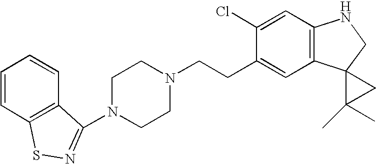

Spiro[cyclopropane-1,3′-{3H}indol]-2′(1′H)-one,5′-[3-[4-{1,2-benzisothiazol-3-yl}-1-piperazinyl]propyl]-6′-chloro-2,2-dimethyl—

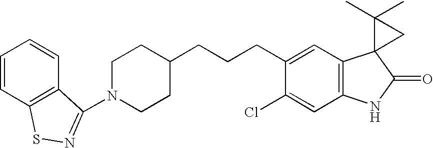

Other preferred embodiments of this invention include compounds of the formula I wherein R⁴ and R⁵ form a spiro 2,2-dimethylcyclopropyl ring.

Other preferred embodiments of this invention include compounds of the formula I wherein R⁴ and R⁵ form an isopropylene group.

Other preferred embodiments of this invention include compounds of the formula I wherein one or both of R² and R³ are hydrogen.

Examples of other embodiments of the present invention are the following compounds and their pharmaceutically acceptable salts:

3-Isopropylidene-5-[2-(4-naphthalen-1-yl-piperazin-1-yl)-ethyl]1,3-dihydro-indol-2-one;

3-Isopropylidene-5-[3-(4-naphthalen-1-yl-piperazin-1-yl)-propyl]1,3-dihydro-indol-2-one;

5-{2-[4-(6-Fluoro-benzo[d]isoxazol-3-yl)-piperazin-1-yl]-ethyl}-3-isopropylidene-1,3-dihydro-indol-2-one;

5-{3-[4-(6-Fluoro-benzo[d]isoxazol-3-yl)-piperazin-1-yl]-propyl}-3-isopropylidene-1,3-dihydro-indol-2-one;

5-{2-[4-(1-Hydroxy-1H-1 lambda*4*-benzo[d]isothiazol-3-yl)-piperazin-1-yl]-ethyl}-3-isopropylidene-1,3-dihydro-indol-2-one;

5-{3-[4-(1-Hydroxy-1H-1 lambda*4*-benzo[d]isothiazol-3-yl)-piperazin-1-yl]-propyl}-3-isopropylidene-1,3-dihydro-indol-2-one;

3-Isopropylidene-5-[2-(4-isoquinolin-1-yl-piperazin-1-yl)-ethyl]-1,3-dihydro-indol-2-one;

3-Isopropylidene-5-[3-(4-isoquinolin-1-yl-piperazin-1-yl)-propyl]-1,3-dihydro-indol-2-one;

5-[2-(4-Benzo[b]thiophen-3-yl-piperazin-1-yl)-ethyl]-3-isopropylidene-1,3-dihydro-indol-2-one;

5-[3-(4-Benzo[b]thiophen-3-yl-piperazin-1-yl)-propyl]-3-isopropylidene-1,3-dihydro-indol-2-one;

5-[2-(4-Benzofuran-3-yl-piperazin-1-yl)-ethyl]-3-isopropylidene-1,3-dihydro-indol-2-one;

5-[3-(4-Benzofuran-3-yl-piperazin-1-yl)-propyl]-3-isopropylidene-1,3-dihydro-indol-2-one;

3-Isopropylidene-5-{2-[4-(4-propenyl-5-vinyl-1H-pyrrol-3-yl)piperazin-1-yl]-ethyl}-1,3-dihydro-indol-2-one;

5-{3-[4-(1H-Indol-3-yl)-piperazin-1-yl]-propyl}-3-isopropylidene-1,3-dihydro-indol-2-one;

5-[2-(4-Benztriazol-1-yl-piperazin-1-yl)-ethyl]-3-isopropylidene-1,3-dihydro-indol-2-one; and

5-{2-[4-(6-Jydroxy-quinolin-8-yl)-piperazin-1-yl]-ethyl}-3-isopropylidene-1,3-dihydro-indol-2-one.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso-sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “aryl”, as used herein, unless otherwise indicated, includes an aromatic ring system with no heteroatoms (e.g., phenyl or naphthyl).

The term “alkoxy”, as used herein, unless otherwise indicated, means “alkyl-O-”, wherein “alkyl” is as defined above. Examples of “alkoxy” groups include, but are not limited to, methoxy, ethoxy, propoxy, butoxy and pentoxy.

The term “alkenyl”, as used herein, unless otherwise indicated, includes unsaturated hydrocarbon radicals having one or more double bonds connecting two carbon atoms, wherein said hydrocarbon radical may have straight, branched or cyclic moieties or combinations thereof. Examples of “alkenyl” groups include, but are not limited to, ethenyl, propenyl, butenyl, pentenyl.

The term “heteroaryl” or as used herein, unless otherwise indicated, includes monocyclic aromatic heterocycles containing five or six ring members, of which from 1 to 4 can be heteroatoms selected, independently, from N, S and O, and bicyclic aromatic heterocycles containing from eight to twelve ring members, of which from 1 to 4 can be heteroatoms selected, independently, from N, S and O.

The term “one or more substituents”, as used herein, refers to a number of substituents that equals from one to the maximum number of substituents possible based on the number of available bonding sites.

The terms “halo” and “halogen”, as used herein, unless otherwise indicated, include, fluoro, chloro, bromo and iodo.

The term “treating”, as used herein, refers to reversing, alleviating, inhibiting the progress of, or preventing the disorder or condition to which such term applies, or preventing one or more symptoms of such condition or disorder.

The term “treatment”, as used herein, refers to the act of treating, as “treating” is defined immediately above.

The term “methylene”, as used herein, means —CH₂—.

The term “ethylene”, as used herein, means —CH₂CH₂—.

The term “propylene”, as used herein, means —CH₂CH₂CH₂—.

The compounds of formula I and their pharmaceutically acceptable salts are also referred to herein, collectively, as the “novel compounds of this invention” and the “active compounds of this invention”.

This invention also relates to a pharmaceutical composition comprising a therapeutically effective amount of a compound of the formula I, or a pharmaceutically acceptable salt thereof, and a pharmaceutically acceptable carrier.

Compounds of formula I may contain chiral centers and therefore may exist in different enantiomeric and diastereomeric forms. This invention relates to all optical isomers and all stereoisomers of compounds of the formula I, both as racemic mixtures and as individual enantiomers and diastereoisomers of such compounds, and mixtures thereof, and to all pharmaceutical compositions and methods of treatment defined above that contain or employ them, respectively. Individual isomers can be obtained by known methods, such as optical resolution, optically selective reaction, or chromatographic separation in the preparation of the final product or its intermediate. Individual enantiomers of the compounds of formula I may have advantages, as compared with the racemic mixtures of these compounds, in the treatment of various disorders or conditions.

In so far as the compounds of formula I of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples below, by substituting a readily available isotopically labelled reagent for a non-isotopically labelled reagent.

The compounds of formula I of this invention have useful pharmaceutical and medicinal properties.

This invention also relates to a method of treating a disorder or condition selected from the group consisting of single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound of the formula I, or a pharmaceutically acceptable salt thereof, that is effective in treating such disorder or condition.

The compounds of formula I and their pharmaceutically acceptable salts are also referred to herein, collectively, as the “novel compounds of this invention” and the “active compounds of this invention”.

This invention also relates to a pharmaceutical composition comprising a therapeutically effective amount of a compound of the formula I, or a pharmaceutically acceptable salt thereof, and a pharmaceutically acceptable carrier.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising an amount of a compound of the formula I, or a pharmaceutically acceptable salt thereof, that is effective in treating such disorder or condition, and a pharmaceutically acceptable carrier.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies.

Another more specific embodiment of this invention relates to the above method wherein the compound of formula I is administered to a human for the treatment of any two or more comorbid disorders or conditions selected from those disorders and conditions referred to in any of the above methods.

For the treatment of depression, anxiety, schizophrenia or any of the other disorders and conditions referred to above in the descriptions of the methods and pharmaceutical compositions of this invention, the novel compounds of this invention can be used in conjunction with one or more other antidepressants or anti-anxiety agents. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and a typical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine and sertraline. Examples of monoamine oxidase inhibitors include isocarboxazid, phenelzine, and tranylcyclopramine. Suitable reversible inhibitors of monoamine oxidase include moclobemide. Suitable serotonin and noradrenaline reuptake inhibitors of use in the present invention include venlafaxine. Suitable CRF antagonists include those compounds described in International Patent Application Nos. WO 94/13643, WO 94/13644, WO 94/13661, WO 94/13676 and WO 94/13677. Suitable a typical anti-depressants include bupropion, lithium, nefazodone, trazodone and viloxazine. Suitable NK-1 receptor antagonists include those referred to in World Patent Publication WO 01/77100.

Suitable classes of anti-anxiety agents that can be used in combination with the active compounds of this invention include benzodiazepines and serotonin IA (5-HT_{IA}) agonists or antagonists, especially 5-HT_{IA} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{IA} receptor agonists or antagonists include buspirone, flesinoxan, gepirone and ipsapirone.

This invention also relates to a method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising administering to said mammal:

(a) a compound of the formula I or a pharmaceutically acceptable salt thereof; and

(b) another pharmaceutically active compound that is an antidepressant or anti-anxiety agent, or a pharmaceutically acceptable salt thereof;

wherein the active compounds “a” and “b” are present in amounts that render the combination effective in treating such disorder or condition.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies.

Another more specific embodiment of this invention relates to the above method wherein the compound of formula I and the additional antidepressant or anti-anxiety agent are administered to a human for the treatment of any two or more comorbid disorders or conditions selected from those disorders and conditions referred to in any of the above methods.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising:

(a) a compound of the formula I or a pharmaceutically acceptable salt thereof;

(b) another pharmaceutically active compound that is an antidepressant or anti-anxiety agent, or a pharmaceutically acceptable salt thereof; and

(c) a pharmaceutically acceptable carrier;

wherein the active compounds “a” and “b” are present in amounts that render the composition effective in treating such disorder or condition.

### DETAILED DESCRIPTION OF THE INVENTION

The compounds of formula I of the present invention may be prepared as described in the following reaction schemes. Unless otherwise indicated, Ar, A, and R¹ through R⁸ in the reaction schemes and discussion that follow are as defined above.

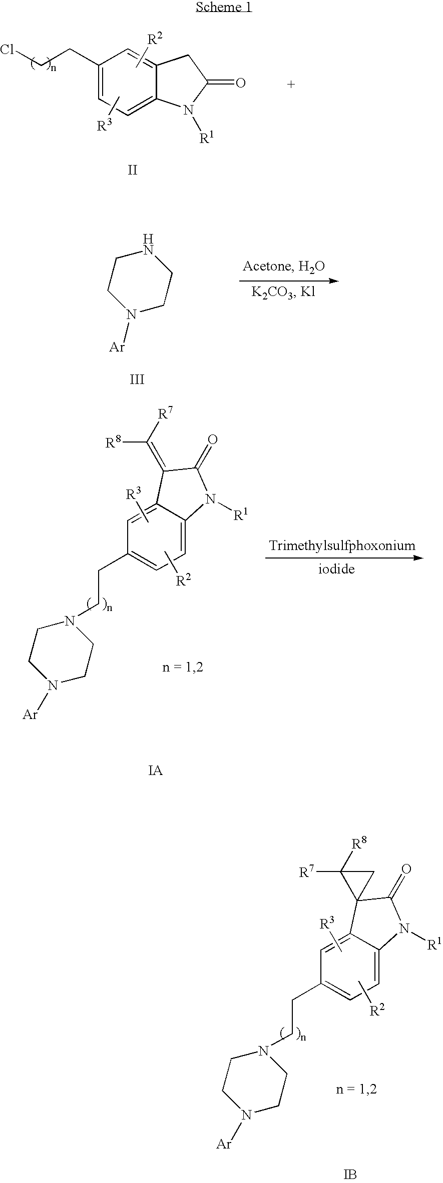

Scheme 1 illustrates the synthesis of compounds of the formula I wherein A is ethylene, propylene or butylene and R⁴ and R⁵ form an olefin terminally substituted with R⁷ and R⁸ wherein R⁷ and R⁸ are methyl (hereinafter referred to as compounds of the formula IA) and compounds of the formula I wherein A is ethylene, propylene or butylene and R⁴ and R⁵ form a 2,2-dimethylspirocyclopropyl group (hereinafter referred to as compounds of the formula IB). Referring to Scheme 1, an oxindole having the formula II is combined with an aryl-piperazinyl compound of the formula III and acetone to yield the corresponding compound of formula IA. This reaction is typically carried out in a polar solvent such as acetonitrile, water, or a lower alcohol, in the presence of a base. Preferably the reaction is carried out in a 2:1 mixture of acetone and water. Suitable bases include sodium and potassium carbonate and sodium and potassium t-butoxide, with potassium carbonate being preferred. It is also preferable to conduct the reaction in the presence of a catalytic amount of potassium iodide. The reaction temperature can range from about 30° C. to about 50° C., and is preferably between about 30° C. and 100° C. Typically, the reaction is carried out for a period ranging from about 2 hours to about 3 days, until such time as the reaction is complete. The product can be isolated by precipitation or an extractive workup.

Alternatively, the condensation with acetone can be carried out as a separate step. This can be accomplished by first reacting the compound of formula II with that of formula III in a polar solvent such as those as described above to form an intermediate having the formula IA-a, which is identical to the compound of formula IA except that R⁴ and R⁵ are hydrogen, and then reacting the compound of formula IA-a, either in situ or after isolation, with acetone in a polar solvent such as those described above, and in the presence of a base such as those described above. Both reactions are typically carried out at a temperature from about 30° C. to about 150° C., preferably between about 30° C. and 100° C., for a period ranging from about 2 hours to about 3 days, until such time as the reaction is complete.

Compounds of the formula IA wherein one or both of R⁷ and R⁸ are other than methyl can be formed using a procedure similar to that described above, but wherein acetone is replaced with the appropriate ketone having the formula R⁷C(═O)R⁸.

Compounds of the formula IB can be prepared in the following manner. The corresponding olefin of formula IA is treated with dimethylsulfoxonium methylide in a suitable dry polar solvent such as dimethylformamide (DMF) or dimethylsulfonamide (DMSO), at a temperature from about −10° C. to about 90° C., preferably at about 25° C., for about 1 to 24 hours until the reaction is done. This reaction is preferably conducted under an inert atmosphere. The product can then be isolated by an extractive workup and purified, if necessary, by column chromatography, recrystallization or salt formation.

The dimethylsulfoxonium methylide used in the above reaction can be generated from the reaction of trimethylsuloxonium iodide or chloride, in a suitable polar solvent such as dry DMF or dry DMSO, with a strong base. The base, which is preferably in solid form, is suitably a metal hydroxide, e.g., sodium hydroxide or lithium hydroxide, or an alkali metal hydride, e.g., sodium hydride. This formation is carried out at a temperature from about −10° C. to about 90° C., preferably at about 25° C. Preferably, it is carried out under an inert atmosphere. It is possible to use a phase transfer catalyst such as tetrabutyl-n-ammonium bromide or the like in the formation of the dimethylsulfoxonium methylide.

Compounds of the formula II can be prepared as described in J. Med. Chem., 1991, 34, 1860-1866, in J. Med. Chem., 1996, 39, 143-148, and in U.S. Pat. No. 4,411,901. The foregoing references are incorporated herein by reference in their entireties. The synthesis of compounds of the formula II wherein n is one, R² and R³ are hydrogen and R¹ is methyl is depicted in Scheme 1a.

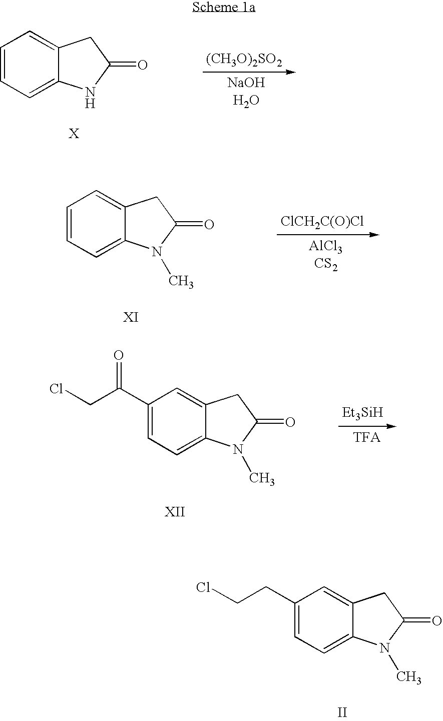

Referring to Scheme 1a, the compound of formula X (oxindole) is reacted with water, sodium hydroxide and dimethoxysulfate, as described in detail in Preparation 1 of the experimental examples, to form the methylated derivative of formula XI. The compound of formula XI is then reacted with chloroacetylchloride, carbon disulfide and anhydrous aluminum chloride, as described in detail in Preparation 2, to form the compound of formula XII. Reaction of the compound of formula XII with triethylsilicon hydride in trifluoroacetic acid, as described in Preparation 3, yields the compound of formula II.

Scheme 2 illustrates the synthesis of compounds of the formula I wherein A is —(CH₂)ₙC(═O)—, —(CH₂)ₙCH(OH)— or —(CH₂)ₙCH(NHR)—. These compounds are hereinafter referred to as compounds of the formula IC, IE and ID, respectively.

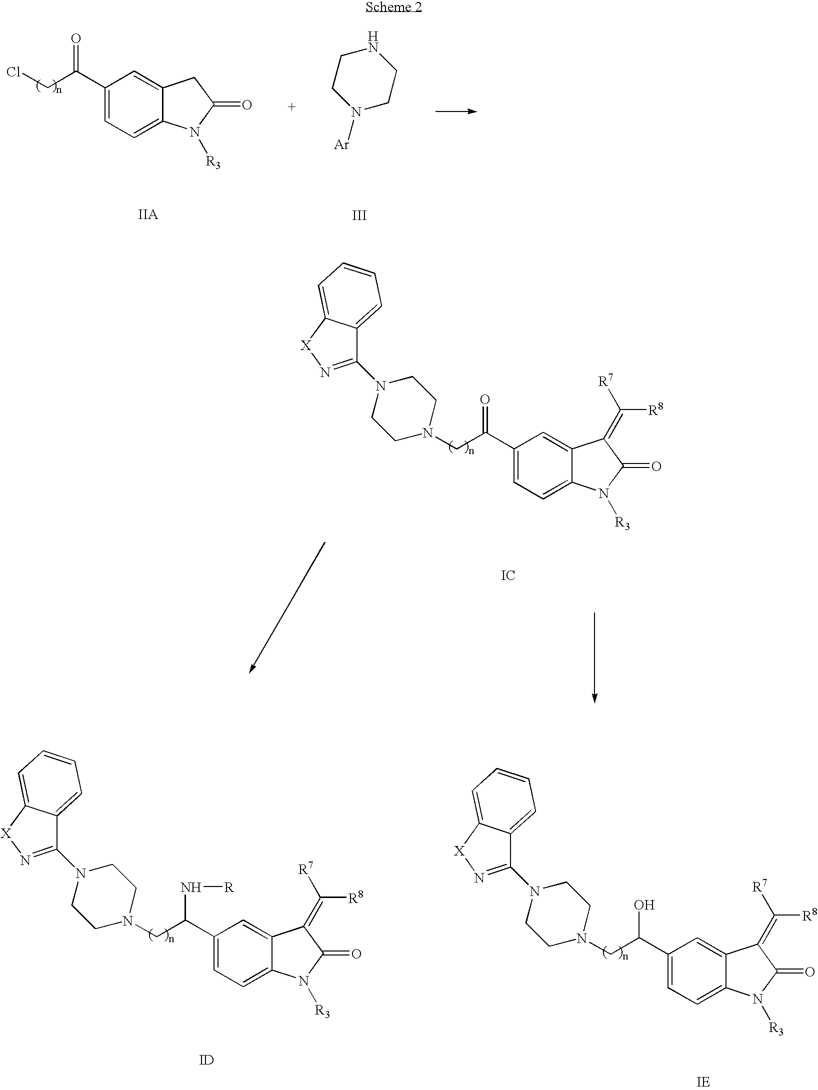

Referring to Scheme 2, the chloro ketone of formula II is combined with an aryl-piperazinyl compound of the formula III to yield the corresponding compound of formula IC. This reaction is typically carried out in a polar solvent such as an alcohol, water or acetonitrile, and a ketone of the formula R⁷C(═O)R⁸ in the presence of a base. Preferably the reaction is carried out in a 2:1 mixture of R⁷C(═O)R⁸ and water. Suitable bases include potassium carbonate, sodium carbonate and potassium t-butoxide, with potassium carbonate being preferred. It is also preferable to conduct the reaction in the presence of a catalytic amount of potassium iodide. The reaction temperature can range from about 30° C. to about 150° C., and is preferably between about 30° C. and 100° C. Typically, the reaction is carried out for a period ranging from about 2 hours to about 3 days, until such time as the reaction is complete. The product can be isolated by precipitation or an extractive workup. If necessary the compound can be purified by column chromatography, using silica gel and eluting with a suitable solvent or solvent mixture.

Alternatively, the condensation with the appropriate ketone of formula R⁷C(═O)R⁸ can be carried out as a second step, as described above in the discussion of the reactions illustrated in Scheme 1.

The compound of formula IC can then be subjected to reduction conditions such as sodium borohydride, sodium cyanoborohydride, and the like to reduce the ketone in the linker chain to an alcohol, thus forming the corresponding compound of formula IE. These reactions are typically carried out in a suitable solvent such as an alcohol or an ether such as THF, at a temperature of about 0° C. to about 80° C., preferably between about 0° C. and 25° C. The reaction is generally carried out from about 5 minutes to 2 days until complete. The resulting compound of formula IE can be purified by column chromatography, recrystallization or salt formation.

Compounds of the formula ID can be obtained by subjecting the corresponding compounds of formula IC to reductive amination conditions using methods well known to those of skill in the art. Typically, this involves treating the compound of formula IC with the appropriate amine to form the intermediate imine, and reducing the imine, either in situ or after isolation, with an appropriate reducing agent such as sodium cyanoborohydride, another suitable hydride reducing agent, or by hydrogenation with an appropriate metal catalyst such as Raney nickel or platinumon carbon or palladium on carbon or palladium, using methods well known to those of skill in the art. The reaction temperature can range from about −10° C. to about 100° C., and is preferably between about 0° C. and about 50° C. Suitable solvents include ethers (e.g., ethyl ether), lower alkanols, and water. The resulting compounds of the formula ID can be purified by column chromatography, recrystallization or salt formation.

Compounds of the formulas IC, ID and IE wherein R⁴ and R⁵ form a substituted or unsubstituted spirocyclic ring can be formed from the corresponding compounds of the formulas IC, ID and IE wherein R⁴ and R⁵ form an olefin, which are depicted above, using the procedure described above for forming compounds of the formula IB from the corresponding compounds of the formula IA.

Scheme 3 illustrates the synthesis of compounds of the formula I wherein A is —(CH₂)ₙNH—. These compounds are hereinafter referred to as compounds of the formula IF.

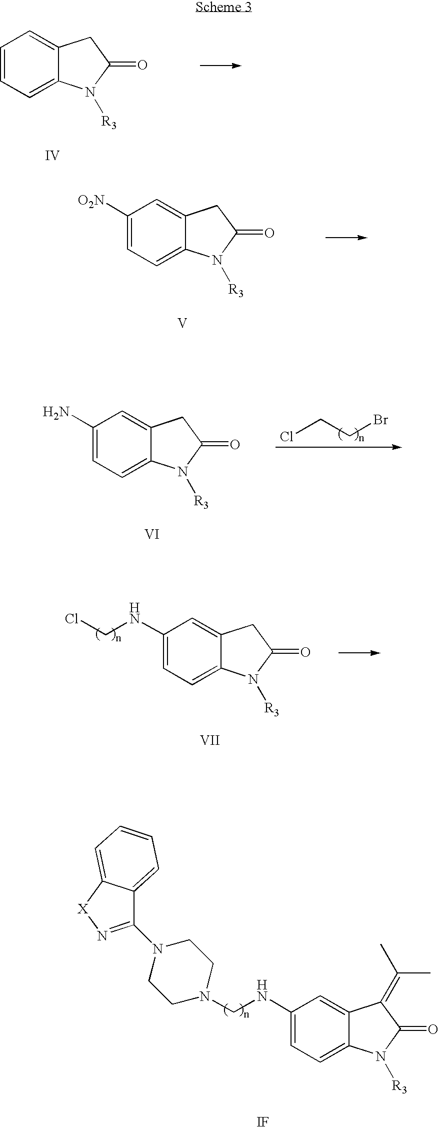

Referring to Scheme 3, a compound of formula IV is nitrated under standard conditions such as nitric acid in sulfuric acid, or ammonium nitrate in trifluoroacetic anhydride, at a temperature from about 0° C. to about 80° C., preferably from about 20° C. to about 50° C., to give the corresponding compound of formula V. The nitro functionality is then reduced, typically by hydrogenation in the presence of a Raney nickel catalyst or other appropriate metal catalyst (e.g., palladium on carbon or platinum on carbon) under a hydrogen pressure of about 1 atmosphere to about 5 atmospheres, in a solvent such as an ether (e.g., ethyl ether), lower alkanol, tetrahydrofuran (THF) or a mixture of two or more of the foregoing solvents (e.g., THF and methanol), using methods well known to those of skill in the art, to afford the corresponding compound of formula VI. This amine is then alkylated with the appropriate chlorobromo alkane in a suitable aprotic solvent such as an ether, lower alkanol, or THF, in the presence of a suitable base such as potassium carbonate present, to give a compound of the formula VII. The alkylation is typically carried out at a temperature from about −10° C. to 100° C., preferably from about 0° C. to about 50° C. Reaction of the compound having structure VII with an aryl-piperazinyl compound of formula III and the appropriate ketone of formula R⁷C(═O)R⁸, under the reaction conditions described above for the formation of compounds of the formula IC yields the corresponding compound of formula IF.

Compounds of the formula IF wherein R⁴ and R⁵ form a substituted or unsubstituted spirocyclic ring can be formed from the corresponding compounds of the formula IF wherein R⁴ and R⁵ form an olefin, which are depicted above, using the procedure described above for forming compounds of the formula IB from the corresponding compounds of the formula IA.

Scheme 4 illustrates the synthesis of compounds of the formula I wherein A is —(CH₂)ₙO—.

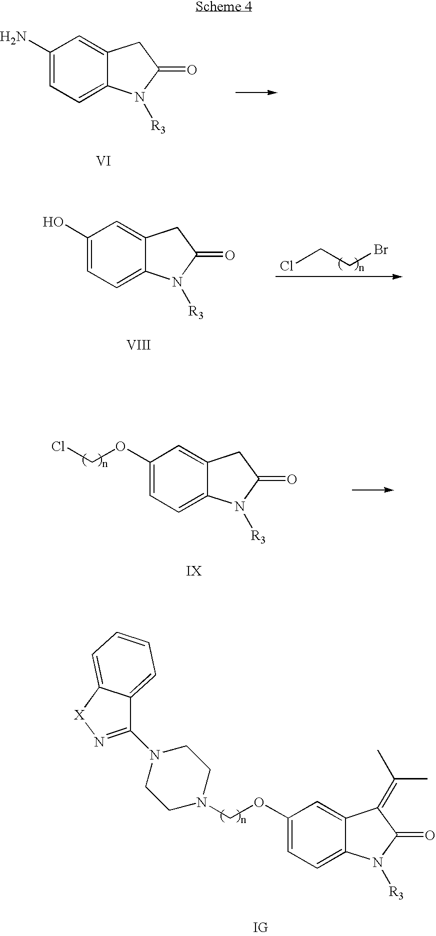

Referring to Scheme 4, ananiline of the formula VI can be converted into the corresponding phenol of formula VIII by formation of an intermediate aryl diazonium ion, preferably generated by treatment with nitrous acid in aqueous solution or by treatment with an alkyl nitrite, followed by hydrolysis. This phenol is then alkylated with the appropriate bromochloro alkane in a suitable aprotic solvent such as an ether, THF, or a lower alkanol, in the presence of a suitable base such as potassium carbonate or cesium carbonate, to give the corresponding compound of formula IX. Reaction of the compound of formula with an aryl-piperazinyl compound of the formula III and an appropriate ketone of the formula R⁷C(═O)R⁸, under the reaction conditions described above for forming compounds of the formula IC yields the corresponding compound of formula IG.

Compounds of the formula IG wherein R⁴ and R⁵ form a substituted or unsubstituted spirocyclic ring can be formed from the corresponding compounds of the formula IG wherein R⁴ and R⁵ form an olefin, which are depicted above, using the procedure described above for forming compounds of the formula IB from the corresponding compounds of the formula IA.

The preparation of other compounds of the formula I and intermediates used in their synthesis that are not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The compounds of the formula I, and the intermediates shown in the above reaction schemes can be isolated and purified by conventional procedures, such as recrystallization or chromatographic separation.

The compounds of the formula I and their pharmaceutically acceptable salts can be administered to mammals via either the oral, parenteral (such as subcutaneous, intraveneous, intramuscular, intrasternal and infusion techniques), rectal, buccal or intranasal routes. In general, these compounds are most desirably administered in doses ranging from about 3 mg to about 600 mg per day, in single or divided doses (i.e., from 1 to 4 doses per day), although variations will necessarily occur depending upon the species, weight and condition of the patient being treated and the patient's individual response to said medicament, as well as on the type of pharmaceutical formulation chosen and the time period and interval at which such administration is carried out. However, a dosage level that is in the range of about 25 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day.

The compounds of the present invention may be administered alone or in combination with pharmaceutically acceptable carriers or diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, suppositories, jellies, gels, pastes, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the weight ratio of the novel compounds of this invention to the pharmaceutically acceptable carrier will be in the range from about 1:6 to about 2:1, and preferably from about 1:4 to about 1:1.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intra-muscular and subcutaneous injection purposes. The preparation of all these solutions under sterile conditions is readily accomplished by standard pharmaceutical techniques well known to those skilled in the art.

This invention relates to methods of treating anxiety, depression, schizophrenia and the other disorders referred to in the description of the methods of the present invention, wherein a novel compound of this invention and one or more of the other active agents referred to above (e.g., an NK1 receptor antagonist, tricyclic antidepressant, 5HT1 D receptor antagonist, or serotonin reuptake inhibitor) are administered together, as part of the same pharmaceutical composition, as well as to methods in which such active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the novel compounds of this invention, when used as a single active agent or in combination with another active agent, will be administered to an adult human in an amount from about 3 mg to about 300 mg per day, in single or divided doses, preferably from about 25 to about 100 mg per day. Such compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of a 5HT reuptake inhibitor, preferably sertraline, in the combination methods and compositions of this invention, for oral, parenteral or buccal administration to the average adult human for the treatment of the conditions referred to above, is from about 0.1 mg to about 2000 mg, preferably from about 1 mg to about 200 mg of the 5HT reuptake inhibitor per unit dose, which could be administered, for example, 1 to 4 times per day. A proposed daily dose of a 5HT1 D receptor antagonist in the combination methods and compositions of this invention, for oral, parenteral, rectal or buccal administration to the average adult human for the treatment of the conditions referred to above, is from about 0.01 mg to about 2000 mg, preferably from about 0.1 mg to about 200 mg of the 5HT1 D receptor antagonist per unit dose, which could be administered, for example, 1 to 4 times per day.

For intranasal administration or administration by inhalation, the novel compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch. Formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

All of the title compounds of the examples were tested and at least one stereoisomer of each such compound exhibited a binding affinity for the D2 receptor, measured as percent inhibition at a concentration of 0.1 μm, of no less than 14% and up to 100%. At least one stereoisomer of each such compound exhibited a binding affinity for the 5HT2 receptor, measured as percent inhibition at a concentration of 0.1 μm, of no less than 80% and up to 100%.

The ability of the compounds of this invention to bind to the dopamine D2 and serotonin 2A (5HT2A) receptors can be determined using conventional radioligand receptor binding assays. All receptors can be heterologously expressed in cell lines and experiments conducted in membrane preparations from the cell lines using procedures outlined below. IC₅₀ concentrations can be determined by nonlinear regression of concentration-dependent reduction in specific binding. The Cheng-Prussoff equation can be used to convert the IC₅₀ to Ki concentrations.

Dopamine D2 Receptor Binding:

[³H]Spiperone binding to a membrane preparation from CHO-hD2L cells is carried out in 250 μl of 50 mM Tris-HCl buffer containing 100 mM NaCl, 1 mM MgCl₂ and 1% DMSO at pH 7.4. Duplicate samples containing (in order of addition) the test compounds, 0.4 nM [³H]spiperone and approximately 12 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-14 were tested using the above assay, in which specific binding determined in the presence of 1 mM haloperidol was 95%. All of the title compounds of Examples 1-14 exhibited Ki values less than or equal to 1 uM. The title compound of Example 1 exhibited a Ki of 14.7 nM. The title compound of Example 5 exhibited a Ki of 1.3 nM. The title compound of Example 4 exhibited a Ki of 49.4 nM.

Serotonin 2A Binding:

[³H] Ketanserin binding to Swiss-h5HT2A cell membranes can be carried out in 250 μl of 50 mM Tris-HCl buffer pH 7.4. Duplicate samples containing (in order of addition) test compounds, 1.0 nM [³H]ketanserin, and approximately 75 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-14 were tested using the above assay, in which specific binding determined in the presence of 1 mM ketanserin was 90%. All of the title compounds of Examples 1-14 exhibited Ki values less than or equal to 1 uM. The title compound of Example 5 exhibited a Ki of 8.4 nM. The title compound of Example 14 exhibited a Ki of 7.45 nM. The title compound of Example 1 exhibited a Ki of 0.75 nM.

The following Examples illustrate the preparation of the compounds of the present invention. Melting points are uncorrected. NMR data are reported in parts per million and are referenced to the deuterium lock signal from the sample solvent.

### EXAMPLES

### Preparation 1

### 1-methyl-1,3-dihydroindol-2-one

To 5 L 4-neck flask (equipped with a mechanical stirrer, condenser and N₂ inlet) was charged with 2 L water and 50% sodium hydroxide (NaOH) (2.52 mol, 201.6 g, 2.25 equiv) followed by oxindole (1.12 mol, 150 g, 1 equiv) and the reaction mixture was heated to ˜40° C. Dimethylsulfate (1.68 mol, 211.7 g (159 mL), 1.5 equiv) was added slowly via syringe. The addition was slightly exothermic with temperature rising to 53° C. When addition was complete, the reaction mixture was heated to ˜100° C. and held for 15 minutes (min). The reaction mixture was cooled to ˜60° C., and a second portion of dimethylsulfate (0.476 mol, 60 g (45 mL), 0.425 equiv) was added. The reaction mixture was heated to ˜100° C. and held 15 min. TLC (heptane/ethyl acetate (EtOAc), 1:1) show methylation was essentially complete. The reaction mixture was cooled to ˜50° C. and the pH adjusted to ˜7 with concentrated HCl. The reaction mixture was seeded, cooled to room temperature and allowed to stand overnight. The solids were collected, wash with water (4×) and dried overnight in a vacuum over at ˜40° C. to give 110.7 g (67%) of 1-methyl-1,3-dihydroindol-2-one as a pink solid, mp 84-86° C.

### Preparation 2

### 5-(2-chloroacetyl)-1-methyl-1,3-dihydroindol-2-one

To a 5 L 4-neck flask (equipped with a mechanical stirrer, condenser, N₂ inlet and NaOH scrubbing solution) was charged sequentially with carbon disulfide (CS₂) (1.5 L), anhydrous aluminum chloride (AlCl₃) (2.7 mol, 359 g, 6 equiv) and chloroacetyl chloride (0.585 mol, 66.1 g (46.6 mL), 1.3 equiv). 1-methyl-1,3-dihydroindol-2-one (0.45 mol, 66.2 g, 1 equiv) was added portionwise over 1 hour; the addition was accompanied with temperature increase of 20→31° C. After the addition of 1-methyl-1,3-dihydroindol-2-one was complete the reaction mixture was stirred 0.75 hour at which time stirring of the reaction mixture was not possible. The reaction mixture was refluxed for 3.5 hours then cooled to room temperature. The carbon disulfide (CS₂) was decanted, the residue cooled in an ice/water bath. The reaction mixture was quenched by very slow addition of ice and water. The resulting tan suspension was stirred overnight at room temperature. The solids was collected, washed with water (4×), dried on the filter for 15 min followed by drying overnight in hood. The product was further dried in a vacuum oven at ˜50° C. for 24 hours; the product was pulverized and dried in a vacuum oven for an additional 3 hours at ˜70° C. to give 91.6 g (91%) of 5-(2-chloroacetyl)-1-methyl-1,3-dihydroindol-2-one of a salmon colored solid, mp 197-200° C.

### Preparation 3

### 5-(2-chloroethyl)-1-methyl-1,3-dihydroindol-2-one

To a 3 L 4-neck flask (equipped with a mechanical stirrer, condenser and a N₂ inlet) was 5-(2-chloroacetyl)-1-methyl-1,3-dihydroindol-2-one (0.673 mol, 150 g, 1 equiv) and TFA (6.73 mol, 767 g (518 mL), 10 equiv) and cooled to ˜8° C. Et₃SiH (1.58 mol, 179.6 g (247 mL) 2.3 equiv) was added slowly over 0.5 hour, maintaining the temperature at 16-18° C. by intermittent cooling and adjusting the rate of addition. When the addition was complete the dark brown reaction solution was allowed to warm on its own to ˜42° C. over 30 minutes. Slight cooling was applied to maintain the reaction temperature at 41-42° C. After ˜20 minutes the temperature began to slowly drop, reaching room temperature over 1.75 hour. The reaction solution was poured into 2.5 L of cold water, the aqueous, oily mixture was seeded and allowed to stir overnight. The solids were collected, washed with water (3×) and heptane (2×). The product was dried on the filter for 1 hour then overnight in a vacuum oven at ˜45° C. to give 132.6 g (94%) of 5-(2-chloroethyl)-1-methyl-1,3-dihydroindol-2-one as a light brown solid, mp 136° C.

### Preparation 4

### 5-(3-chloro-propionyl)-1,3-dihydro-indol-2-one

Aluminum chloride (20.027 g, 150.2 mmol) was suspended in carbon disulfide (100 ml). To this 3-chloropropionyl chloride (4.66 ml, 48.81 mmol) was added. After 10 minutes, 1,3-dihydro-indol-2-one (5.0 g, 37.55 mmol) was added slowly to the reaction. The mixture was heated to reflux and stirred for 3 hours. After cooling carbon disulfide was decanted off and the reaction flask was cooled in an ice bath. Ice and water was slowly added until all the aluminum chloride had reacted and a precipitate had formed. Stirred overnight. The precipitate was filtered off and washed with ample amounts of water. The resultant solid 5-(3-chloro-propionyl)-1,3-dihydroindol-2-one was dried in vacuo to afford 8.24 g. Yield 98%; MS (APCI): 224 [M+H]⁺.

### Preparation 5

### 5-(3-chloro-propyl)-1,3-dihydro-indol-2-one

5-(3-chloro-propionyl)-1,3-dihydro-indol-2-one (8.24 g, 36.94 mmol) was dissolved in triflouroacetic acid (28.46 ml, 396.4 mmol). Triethylsilane (13.57 ml, 84.96 mmol) was added slowly to the mixture and was stirred overnight. An ice water/hexane (10:1) mixture was added to the reaction and was stirred vigorously for 1 hour. The resultant precipitate was filtered off, washed with water and dried in vacuo to afford 5-(3-chloro-propyl)-1,3-dihydro-indol-2-one. Yield 94%; MS (APCI): 210 [M+H]⁺.

### Preparation 6

### 6-chloro-5-(3-chloro-propyl)-1,3-dihydro-indol-2-one

The title compound can be prepared using a method analogous to that described in Preparation 4, starting with 1,3-dihydro-6-chloro-indol-2-one.

### Preparation 7

### 3-piperazin-1-yl-1,2-benzisoxazole

A two liter reactor was charged with the chloro-oxazole compound (95 g, 0.619), piperazine (236 g 2.74 m) and acetonitrile (500 mL). The reactor was sealed, stirred and slowly warmed to an internal temperature of 130° C. then maintained at that temperature for 5 hours. The reactor was cooled, vented, and rinsed with water. The reaction was worked up in the usual manner to afford the product.

### Preparation 8

### 3-piperazin-1-yl-1H-indazole

A neat mixture of 3-chloroindazole (15.72 g, 0.103 mol) and piperazine (46.0 g, 0.534 mol) is heated at 250° C. for 14 hours in a stainless steel sealed vessel. Upon cooling to room temperature, the viscous residue is partitioned between aqueous 1.0N sodium hydroxide (NaOH) and methylene chloride. The organic layer is dried over magnesium sulfate, filtered, and the filtrate treated with 4N hydrochloric acid (HCl) in dioxane, which results in the precipitation of the product as a gummy residue. This is taken up in water to precipitate side-products, and the filtrate re-concentrated to afford the title compound. 19.03 g (77%) MS (APCI), m+1=203; m−1=201.

### Example 1

### 5-[2-(4-1,2-benzisothiazol-3-yl-piperazin-1-yl)-ethyl]-3-isopropylidene-1-methyl-1,3-dihydro-indol-2-one

5-(2-Chloro-ethyl)-1-methyl-1,3-dihydro-indol-2-one (10.00 g, 47.83 mmol), and benzo[d]-isothiazol-3-yl-piperazin-1-yl (20.98 g, 95.66 mmol) were added dry in a flask. This was suspended in a 1:1 mixture of acetone/water (150 ml). −325 mesh potassium carbonate (26.48 g, 191.32 mmol) was then added to the mixture, followed by a catalytic amount of potassium iodide. The mixture was refluxed at 100° C. for 2 days. The solid precipitate was filtered and washed with water and acetonitrile. This resulted in the title compound as a pure yellow solid.

Yield 79%; MS (APCI): 433 [M+H]⁺.

### Example 2

### 5-[2-(4-1,2-benzisothiazol-3-yl-piperazin-1-yl)-ethyl]-3-isopropylidene-1,3-dihydro-indol-2-one

5-(2-Chloro-ethyl)-1,3-dihydro-indol-2-one (2.0 g, 10.25 mmol) and 1,2-benzisothiazol-3-yl-piperazin-1-yl (4.5 g, 20.5 mmol) were suspended in a 1:1 mixture of acetone/water (60 ml). To this −325 mesh potassium carbonate (5.66 g, 41.00 mmol) was added, followed by a catalytic amount of potassium iodide. The mixture was refluxed at 100° C. for 1.5 days. The solid precipitate was filtered and washed with water and acetonitrile. This resulted in a pure yellow solid, 5-[2-(4-1,2-benzisothiazol-3-yl-piperazin-1-yl)-ethyl]-3-isopropylidene-1,3-dihydro-indol-2-one. Yield 56%; MS (APCI): 419 [M+H]⁺.

### Example 3

### 5-[3-(4-1,2-benzisothiazol-3-yl-piperazin-1-yl)-propyl]-3-isopropylidene-1,3-dihydro-indol-2-one

5-(3-chloro-propyl)-1,3-dihydro-indol-2-one (2.0 g, 9.53 mmol) and 1,2-Benzisothiazol-3-yl-piperazin-1-yl (4.18 g, 19.08 mmol) were suspended in a 1:1 mixture of acetone/water (40 ml). To this −325 mesh potassium carbonate (5.27 g, 38.16 mmol) was added and the mixture was refluxed at 100° C. for 2 days. The solid precipitate was filtered and washed with water and acetonitrile. This resulted in a pure yellow solid 5-[3-(4-1,2-Benzisothiazol-3-yl-piperazin-1-yl)-propyl]-3-isopropylidene-1,3-dihydroindol-2-one. Yield 56%; MS (APSI): 433 [M+H]⁺.

### Example 4

### 5-[3-(4-1,2-Benzisoxazol-3-yl-piperazin-1-yl)-propyl]-3-isopropylidene-1,3-dihydro-indol-2-one

5-(3-chloro-propyl)-1,3-dihydro-indol-2-one (0.200 g, 0.954 mmol) and 1,2-enzisoxazol-3-yl-piperazin-1-yl (0.388 g, 1.908 mmol) were suspended in a 1:1 mixture of acetone/water (20 ml). To this −325 mesh potassium carbonate (0.526 g, 3.816 mmol) was then added to the mixture. The mixture was refluxed at 100° C. for 2 days. The solid precipitate was filtered and washed with water and acetonitrile. This resulted in a pure solid 5-[3-(4-1,2-benzisoxazol-3-yl-piperazin-1-yl)-propyl]-3-isopropylidene-1,3-dihydro-indol-2-one. Yield 14%; MS (APCI): 417 [M+H]⁺.

### Example 5

### 5-[2-(4-1,2-benzisothiazol-3-yl-piperazin-1-yl)-ethyl]-6-chloro-3-isopropylidene-1,3-dihydro-indol-2-one

6-Chloro-5-(2-chloro-ethyl)-1,3-dihydro-indol-2-one (1.00 g, 4.34 mmol) and 1,2-Benzisothiazol-3-yl-piperazin-1-yl (1.90 g, 8.69 mmol) were suspended in a 1:1 mixture of acetone/water (50 ml). To this −325 mesh potassium carbonate (2.39 g, 17.36 mmol) was then added to the mixture. The mixture was refluxed at 100° C. for 1 day. The solid precipitate was filtered and washed, but was not product. The filtrate was extracted with EtOAc, dried with MgSO₄ and concentrated. The crude product was purified on MPLC ethyl acetate (EtOAc) to afford 5-[2-(4-1,2-Benzisothiazol-3-yl-piperazin-1-yl)-ethyl]-6-chloro-3-isopropylidene-1,3-dihydro-indol-2-one. Yield 3%; MS (APCI): 453 [M+H]⁺.

### Example 6

### 5-[2-(4-1,2-benzisothiazol-3-yl-piperazin-1-yl)-ethyl]-6-chloro-3-isopropylidene-1,3-dihydro-indol-2-one

Ziprasidone Hydrochloride (5.00 g, 12.14 mmol) (U.S. Pat. No. 5,206,366) was suspended in a 1:1 mixture of acetone/water (60 ml). To this −325 mesh potassium carbonate (4.20 g, 30.27 mmol) was then added to the mixture. The mixture was refluxed at 100° C. for 1 day. Little product had formed. Another 2.5 equivalents of potassium carbonate (4.18, 30.35 mmol) was added. The reaction went to completion overnight. The pink solid was filtered and washed with water and acetonitrile to afford the pure product 5-[2-(4-1,2-benzisothiazol-3-yl-piperazin-1-yl)-ethyl]-6-chloro-3-isopropylidene-1,3-dihydro-indol-2-one. Yield 85%; MS (APCI): 453 [M+H]⁺.

### Example 7

### 5-[2-(4-1,2-benzisothiazol-3-yl-piperazin-1-yl)-propyl]-6-chloro-3-isopropylidene-1,3-dihydro-indol-2-one

6-Chloro-5-(2-chloro-propyl)-1,3-dihydro-indol-2-one (1.00 g, 4.09 mmol) and 1,2-Benzisothiazol-3-yl-piperazin-1-yl (1.79 g, 8.19 mmol) were suspended in a 1:1 mixture of acetone/water (50 ml). To this −325 mesh potassium carbonate (2.26 g, 16.38 mmol) was then added to the mixture. The mixture was refluxed at 100° C. for 1 day. The filtrate was taken up in EtOAc, washed three times with water, sat'd NaCl, dried with MgSO₄ and concentrated. MPLC (4:1) ethyl acetate/hexane (EtOAc/Hex) resulted in 90% pure solid. This was washed several times with acetonitrile to yield pure product 5-[2-(4-1,2-benzisothiazol-3-yl-piperazin-1-yl)-propyl]-6-chloro-3-isopropylidene-1,3-dihydro-indol-2-one. Yield 22%; MS (APCI): 467 [M+H]⁺.

### Example 8

### 5-[3-(4-1,2-benzisoxazol-3-yl-piperazin-1-yl)-ethyl]-3-isopropylidene-1,3-dihydro-indol-2-one

5-(3-chloro-ethyl)-1,3-dihydro-indol-2-one (1.0 g, 4.9 mmol) and 1,2-benzisoxazol-3-yl-piperazin-1-yl (1.0 g, 4.9 mmol) were suspended in a 1:1 mixture of acetone/water (40 ml). To this −325 mesh potassium carbonate (1.69 g, 12.25 mmol) was then added to the mixture. The mixture was refluxed at 100° C. for 1 day. The solid precipitate was filtered and washed, but was discarded. The filtrate was extracted with EtOAc, dried with MgSO₄ and concentrated. The solid was then washed with acetonitrile to result in pure product 5-[3-(4-1,2-Benzisoxazol-3-yl-piperazin-1-yl)-ethyl]-3-isopropylidene-1,3-dihydro-indol-2-one. Yield 2%; MS (APSI): 403 [M+H]⁺.

### Example 9

### 5-{3-[4-(1H-indazol-3-yl)-piperazin-1-yl]-propyl}-3-isopropylidene-1,3-dihydro-indol-2-one

5-(3-chloro-propyl)-1,3-dihydro-indol-2-one (2.0 g, 9.53 mmol) and Piperizinal-Indazole (1.93 g, 9.54 mmol) were suspended in a 1:1 mixture of acetone/water (60 ml). To this −325 mesh potassium carbonate (3.29 g, 23.85 mmol) was then added to the mixture. The mixture was refluxed at 100° C. for 3 days. The residue was taken up in EtOAc. The organic was washed with water, sat'd NaCl, dried with MgSO₄ and concentrated. The crude product was purified on MPLC (4/1) EtOAc/Hex, (98/2) CH₂Cl₂/MeOH and finally flushed with MeOH to result in 90% pure compound. The solid washed with ample amounts of acetonitrile to the the product 5-{3-[4-(1H-Indazol-3-yl)-piperazin-1-yl]-propyl}-3-isopropylidene-1,3-dihydro-indol-2-one. Yield 4%; MS (APCI): 416 [M+H]⁺.

### Example 10

### Spiro[cyclopropane-1,3′-{3H}indol]-2′(1′H)-one,5′-[2-[4-{1,2-benzisothiazol-3-yl}-1-piperazinyl]ethyl]-1′,2,2-trimethyl—

Trimethylsulfoxonium iodide (0.761 g, 3.46 mmol) was stirred in anhydrous DMF, under a nitrogen (N₂) atmosphere. To this suspension, sodium hydride (NaH) [60% disp.] (0.138 g, 3.46 mmol) was added and the reaction was allowed to stir for 20 min. After cooling to 0° C., 5-[2-(4-1,2-benzisothiazol-3-yl-piperazin-1-yl)-ethyl]-3-isopropylidene-1-methyl-1,3-dihydro-indol-2-one (1.00 g, 2.31 mmol) Example 1 was added by suspending in anhydrous dimethylformamide (DMF) and pipetting it to the reaction slowly. The reaction was allowed to warm to room temp and was stirred overnight. The reaction was diluted with EtOAc. The organic layer was washed with ample amounts of water, sodium chloride (NaCl), dried with MgSO₄ and concentrated. The crude product was purified on MPLC (4/1 EtOAc/Hexanes). Yield 64% spiro[cyclopropane-1,3′-{3H}indol]-2′(1′H-one,5′-[2-[4-{1,2-benzisothiazol-3-yl}-1-piperazinyl]ethyl]-1′,2,2-trimethyl-; MS (APCI): 447 [M+H]⁺.

### Example 11

### Spiro[cyclopropane-1,3′-{3H}indol]-2′(1′H)-one,5′-[2-[4-{1,2-benzisothiazol-3-yl}-1-piperazinyl]ethyl]-2,2-di methyl—

Trimethylsulfoxonium iodide (0.790 g, 3.59 mmol) was stirred in anhydrous DMF, under N₂ (atm). To this suspension, NaH [60% disp.] (0.1436 g, 3.59 mmol) was added and the reaction was allowed to stir for 20 min. At 0° C., 5-[2-(4-1,2-Benzisothiazol-3-yl-piperazin-1-yl)-ethyl]-3-isopropylidene-1,3-dihydro-indol-2-one (3.37 g, 15.38 mmol) Example 2 was added by suspending in anhydrous DMF and pipetting it to the reaction slowly. The reaction was allowed to warm to room temp and was stirred overnight. The reaction was diluted with EtOAc. The organic layer was washed with ample amounts of water, NaCl, dried with MgSO₄ and concentrated and dried in vacuo to afford Spiro-[cyclopropane-1,3′-{3H}indol]-2′(1′H)-one,5′-[2-[4-{1,2-benzisothiazol-3-yl}-1-piperazinyl]ethyl]-2,2-dimethyl—. No purification was needed. Yield 75%; MS (APCI): 433 [M+H]⁺.

### Example 12

### Spiro[cyclopropane-1,3′-{3H}indol]-2′(1′H)-one,5′-[3-[4-{1,2-benzisothiazol-3-yl}-1-piperazinyl]propyl]-2,2-dimethyl—

Trimethylsulfoxonium iodide (0.764 g, 3.47 mmol) was stirred in anhydrous DMF, under a nitrogen (N₂) atmosphere. To this suspension, NaH [60% disp.] (0.139 g, 3.47 mmol) was added and the reaction was allowed to stir for 20 min. 5-[3-(4-1,2-Benzisothiazol-3-yl-piperazin-1-yl)propyl]-3-isopropylidene-1,3-dihydro-indol-2-one (1.0 g, 2.31 mmol) Example 3 was added by suspending in anhydrous DMF and pipetting it to the reaction slowly. The reaction was stirred overnight. The reaction was diluted with EtOAc. The organic layer was washed with ample amounts of water, sodium chloride (NaCl), dried with MgSO₄ and concentrated and dried in vacuo to afford spiro[cyclopropane-1,3′-{3H}indol]-2′(1′H)-one,5′-[3-[4-{1,2-benzisothiazol-3-yl}-1-piperazinyl]propyl]-2,2-dimethyl-. The crude product was purified on MPLC (EtOAc). Yield 45%; MS (APCI): 447 [M+H]⁺.

### Example 13

### Spiro[cyclopropane-1,3′-{3H}indol]-2′(1′H)-one,5′-[2-[4-{1,2-benzisothiazol-3-yl}-1-piperazinyl]ethyl]-6′-chloro-2,2-dimethyl—

Trimethylsulfoxonium iodide (1.08 g, 4.96 mmol) was stirred in anhydrous DMF, under N₂ (atm). To this suspension, sodium hydride (NaH) [60% disp.] (0.20 g, 4.96 mmol) was added and the reaction was allowed to stir for 20 min. 5-[2-(4-1,2-Benzisothiazol-3-yl-piperazin-1-yl)ethyl]-6-chloro-3-isopropylidene-1,3-dihydro-indol-2-one (1.50 g, 3.31 mmol) was added by suspending in anhydrous DMF and pipetting it to the reaction slowly. The reaction was stirred overnight. The reaction was diluted with ethyl acetate (EtOAc). The organic layer was washed with ample amounts of water, NaCl, dried with magnesium sulfate (MgSO₄) and concentrated. The impure solid was taken up in acetonitrile and was washed thoroughly. The impure solid was taken up in methylene chloride (CH₂Cl₂) and stirred for 1 hour, then filtered. This resulted in the pure product spiro[cyclopropane-1,3′-{3H}indol]-2′(1′H)-one,5′-[2-[4-{1,2-benzisothiazol-3-yl}-1-piperazinyl]ethyl]-6′-chloro-2,2-dimethyl-. Yield 43%; MS (APCI): 467 [M+H]⁺.

### Example 14

### Spiro[cyclopropane-1,3′-{3H}indol]-2′(1′H)-one,5′-[3-[4-{1,2-benzisothiazol-3-yl}-1-piperazinyl]propyl]-6′-chloro-2,2-dimethyl—

Trimethylsulfoxonium iodide (0.56 g, 2.58 mmol) was stirred in anhydrous DMF, under a N₂ atmosphere. To this suspension, NaH [60% disp.] (0.10 g, 2.58 mmol) was added and the reaction was allowed to stir for 20 min. 5-[2-(4-1,2-Benzisothiazol-3-yl-piperazin-1-yl)-propyl]-6-chloro-3-isopropylidene-1,3-dihydro-indol-2-one (0.80 g, 1.72 mmol) Example 7 was added by suspending in anhydrous DMF and pipetting it to the reaction slowly. The reaction was stirred overnight. The reaction was diluted with EtOAc. The organic layer was washed with ample amounts of water, NaCl, dried with MgSO₄ and concentrated. The crude product was purified on MPLC (EtOAc). This resulted in the pure product spiro[cyclopropane-1,3′-{3H}indol]-2′(1′H)-one,5′-[3-[4-{1,2-benzisothiazol-3-yl}-1-piperazinyl]propyl]-6′-chloro-2,2-dimethyl-. Yield 58%; MS (APCI): 481 [M+H]⁺.

1. The present invention relates to compounds of the formula I 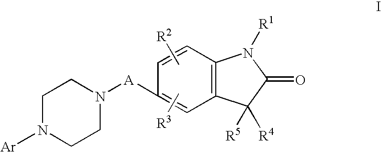
wherein Ar is 1,2-benzisothiazoyl, 1,2-benzisothiazoyl-1-oxide, 1,2-benzisothiazoyl-1-dioxide, 1,2-benzisoxazoyl, naphthyl, pyridyl, quinolyl, isoquinolyl, benzothiadiazolyl, benzotriazolyl, benzoxazolyl, benzoxazolonyl, phthalazinyl, indolyl, indanyl, 1H-indazoyl, or 3-indazolyl, and wherein Ar can optionally be substituted by one or more substituents, preferably from zero to three substituents, independently selected from halo, preferably chloro or fluoro, cyano, nitro, (C₁-C₆) alkyl optionally substituted with from one to three fluorine atoms and (C₁-C₆) alkoxy optionally substituted with from one to three fluorine atoms; with the proviso that Ar can not be attached to the piperizine ring via a phenyl ring of Ar;
A is —(CH₂)ₙCH₂—, wherein n is an integer from one to three, wherein one of the CH₂ groups of A that is not adjacent to the piperizine nitrogen can optionally be replaced by an oxygen atom or by NR, wherein R is (C₁-C₆) alkyl, and wherein one of the carbon atoms of A can optionally be substituted by oxo, amino, NHR wherein R is hydroxy or (C₁-C₆) alkyl, and wherein each R group in a compound of the formula I is independent of any other R group in such compound;
R² and R³ are independently selected from hydrogen, (C₁-C₆) alkyl optionally substituted with from one to three fluorine atoms, (C₁-C₆) alkoxy optionally substituted with from one to three fluorine atoms, (C₂-C₆) alkenyl optionally substituted with from one to three fluorine atoms, (C₂-C₆) alkenoxy optionally substituted with from one to three fluorine atoms, —C(C═O)—(C₁-C₆)alkyl, —C(C═O)—(C₁-C₆)alkenyl having one or two sites of unsaturation, halogen, nitro, cyano, hydroxy, amino, (C₁-C₆) alkylamino, di-(C₁-C₆) alkylamino, aryl and heteroaryl, and wherein said aryl and heteroaryl groups can optionally be substituented with one or more substituents, preferably from zero to two substutuents, independently selected from halo, oxo, nitro, amino, cyano, (C₁-C₆) alkyl optionally substituted with from one to three fluorine atoms and (C₁-C₆) alkoxy optionally substituted with from one to three fluorine atoms;
R¹ is hydrogen, (C₁-C₄) alkyl optionally substituted with from one to three fluorine atoms, aryl, —C(O)R⁶ wherein R⁶ is aryl, (C₁-C₄) alkyl, or aryl-(C₁-C₄) alkyl-, and wherein the alkyl moieties of the aryl-(C₁-C₄) alkyl- and heteroaryl-(C₁-C₄) alkyl groups can be optionally substituted with from one to three fluoro atoms, and wherein the aryl and heteroaryl moieties of these groups can optionally be substituted with one or more substituents, preferably with from zero to two substituents, independently selected from halo, nitro, amino, cyano, (C₁-C₆) alkyl optionally substituted with from one to three fluorine atoms and (C₁-C₆) alkoxy optionally substituted with from one to three fluorine atoms;
R⁴ and R⁵ together represent an olefin optionally terminally substituted by one or two substituents, R⁷ and R⁸, which are independently selected from the group of substituents set forth above in the definition of R² and R³;
or R⁴ and R⁵, taken together, can form a spiro saturated ring containing from 3 to 6 carbon atoms, wherein said ring can be optionally substituted by one or two substituents, R⁷ and R⁸, which are independently selected from the group of substituents set forth above in the definition of R² and R³;
with the proviso that when Ar is benzisothiazol-3-yl, and A is ethylene, and R¹ is hydrogen or unsubstituted (C₁-C₄)alkyl, and R² is hydrogen, halo or methyl, and R³ is hydrogen, halo, nitro, amino, cyano, or substituted or unsubstituted alkyl or substituted or unsubstituted alkoxy; then R⁴ and R⁵ cannot form either a spiro (C₄-C₆)cycloalkyl group or an olefin terminally substituted with R⁷ and R⁸ wherein R⁷ is hydrogen and R⁸ is phenyl;
or a pharmaceutically acceptable salt of such compound.

2. A compound according to claim 1 wherein Ar is a substituted or unsubstituted bicyclic ring system selected from the following: 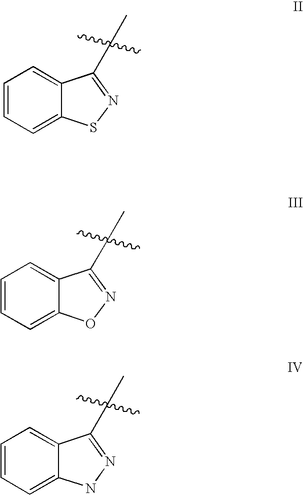
and wherein A is —CH₂—, —CH₂—CH₂—, —(C═O)—, —CH₂(C═O)—, —CH(OH)—, —CH₂—CH(OH)—, —CH—N(R)—, or —CH₂—CH—N(R)—, and wherein the oxindole moiety attached to A is selected from the following: 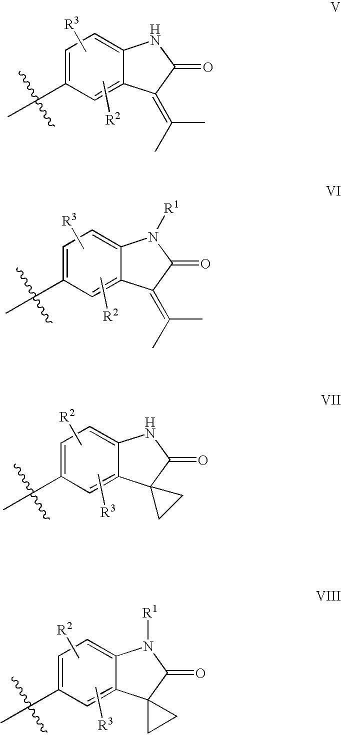
wherein R¹, R² and R³ are as defined above and wherein the spirocyclopropyl groups can be substituted or unsubstituted.

3. A compound according to claim 1 wherein R⁴ and R⁵ form a spiro 2,2-dimethylcyclopropyl ring.

4. A compound according to claim 1 wherein R⁴ and R⁵ form an olefin that is optionally terminally substituted with R⁷ and R⁸.

5. A compound according to claim 1 that is selected from the following compounds and their pharmaceutically acceptable salts:
5-[2-(4-1,2-Benzisothiazol-3-yl-piperazin-1-yl)-ethyl]-3-isopropylidene-1-methyl-1,3-dihydro-indol-2-one;
5-[2-(4-1,2-Benzisothiazol-3-yl-piperazin-1-yl)-ethyl]-3-isopropylidene-1,3-dihydro-indol-2-one;
5-[3-(4-1,2-Benzisothiazol-3-yl-piperazin-1-yl)-propyl]-3-isopropylidene-1,3-dihydro-indol-2-one;
{5-[3-(4-1,2-Benzisothiazol-3-yl-piperazin-1-yl)-propyl]-3-isopropylidene-2-oxo-2,3-dihydro-indol-1-yl}-acetic acid;
5-[3-(4-1,2-Benzisoxazol-3-yl-piperazin-1-yl)-propyl]-3-isopropylidene-1,3-dihydro-indol-2-one;
5-[2-(4-1,2-Benzisothiazol-3-yl-piperazin-1-yl)-ethyl]-6-chloro-3-isopropylidene-1,3-dihydro-indol-2-one;
5-[2-(4-1,2-Benzisothiazol-3-yl-piperazin-1-yl)-propyl]-6-chloro-3-isopropylidene-1,3-dihydro-indol-2-one;
5-[3-(4-1,2-Benzisoxazol-3-yl-piperazin-1-yl)-ethyl]-3-isopropylidene-1,3-dihydro-indol-2-one;
5-{3-[4-(1H-Indazol-3-yl)-piperazin-1-yl]-propyl}-3-isopropylidene-1,3-dihydro-indol-2-one;
Spiro[cyclopropane-1,3′-{3H}indol]-2′(1′ H)-one,5′-[2-[4-{1,2-benzisothiazol-3-yl}-1-piperazinyl]ethyl]-1′,2,2-trimethyl
Spiro[cyclopropane-1,3′-{3H}indol]-2′(1′H)-one,5′-[2-[4-{1,2-benzisothiazol-3-yl}-1-piperazinyl]ethyl]-2,2-dimethyl—
Spiro[cyclopropane-1,3′-{3H}indol]-2′(1′-one,5′-[3-[4-{1,2-benzisothiazol-3-yl}-1-piperazinyl]propyl]-2,2-dimethyl—
Spiro[cyclopropane-1,3′-{3H}indol]-2′(1′-one,5′-[2-[4-{1,2-benzisothiazol-3-yl}-1-piperazinyl]ethyl]-6′-chloro-2,2-dimethyl—
Spiro[cyclopropane-1,3′-{3H}indol]-2′(1′H)-one,5′-[3-[4-{1,2-benzisothiazol-3-yl}-1-piperazinyl]propyl]-6′-chloro-2,2-dimethyl—

6. A compound according to claim 1 wherein one or both of R² and R³ are hydrogen.

7. A pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising an amount of a compound according to claim 1, or a pharmaceutically acceptable salt thereof, that is effective in treating such disorder or condition, and a pharmaceutically acceptable carrier.

8. A method for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound according to claim 1, or a pharmaceutically acceptable salt thereof, that is effective in treating such disorder or condition.

9. A method according to claim 8, wherein the disorder or condition that is being treated is selected from major depression, single episode depression, recurrent depression, child abuse induced depression, postpartum depression, dysthymia, cyclothymia and bipolar disorder.

10. A method according to claim 8, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

11. A method according to claim 8, wherein the disorder or condition that is being treated is selected from autism, pervasive development disorder, and attention deficit hyperactivity disorder.

12. A method according to claim 8, wherein the disorder or condition that is being treated is selected from generalized anxiety disorder, panic disorder, obsessive-compulsive disorder, post-traumatic stress disorder, and phobias, including social phobia, agoraphobia, and specific phobias.

13. A method according to claim 8, wherein the disorder or condition being treated is schizophrenia with concomitant depression.

14. A method according to claim 8, wherein the disorder or condition being treated is schizophrenia with concomitant anxiety.

15. A method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple aetiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to said mammal:
(a) a compound according to claim 1 or a pharmaceutically acceptable salt thereof; and
(b) another pharmaceutically active compound that is an antidepressant or an anti-anxiety agent, or a pharmaceutically acceptable salt thereof;
wherein the active agents “a” and “b” are present in amounts that render the combination effective in treating such disorder or condition.
